(19) United States
(12) Patent Application Publication
(10) Pub. No.: US 2024/0182843 A1
(43) Pub. Date: Jun. 6, 2024

(54) VARIABLE-VOLUME CHEMOSTAT AND APPLICATIONS THEREOF

(71) Applicant: VANDERBIL UNIVERSITY, Nashville, TN (US)

(72) Inventors: John P. Wikswo, Brentwood, TN (US); Ronald S. Reiserer, Nashville, TN (US); Kyle Hawkins, Nashville, TN (US)

(21) Appl. No.: 18/527,801

(22) Filed: Dec. 4, 2023

Related U.S. Application Data
(60) Provisional application No. 63/429,691, filed on Dec. 2, 2022.

(51) Int. Cl. C12M 1/34; C12M 1/00; C12M 1/06

(52) U.S. Cl. CPC C12M 41/32; C12M 27/06; C12M 29/14; C12M 29/20; C12M 41/34; C12M 41/42; C12M 41/44

(57) ABSTRACT

A chemostat comprises a chamber containing media with cells; an input tube for delivering nutrient-laden media that support cell growth and division within the chamber at an inflow rate; and an output tube for withdrawing a sample from the chamber at an outflow rate. The inflow and outflow rates are regulated such that the chemostat is operable in a sample accumulation phase in which the outflow rate is zero and the inflow rate increases in proportion to an instantaneous volume of media in the chamber so as to accumulate the sample without changes in a metabolic state within the chamber, or a sample withdrawal phase in which the outflow rate is regulated at a higher rate than the inflow rate to withdraw the accumulated sample from the chamber rapidly at one time and the inflow rate remains in proportion to the instantaneous volume to maintain chemostasis in the chamber.

### CROSS-REFERENCE TO RELATED PATENT APPLICATION

This application claims priority to and the benefit of U.S. Provisional Patent Application Ser. No. 63/429,691, filed Dec. 2, 2022, which is incorporated herein by reference in its entirety.

### STATEMENT AS TO RIGHTS UNDER FEDERALLY-SPONSORED RESEARCH

This invention was made with government support under Grant No. 2117782 awarded by the National Science Foundation. The government has certain rights in the invention.

### FIELD OF THE INVENTION

The invention relates generally to bioreactors, and more particularly to a variable-volume chemostat and applications thereof.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

There is an ever-growing interest in the use of bioreactors filled with living cells to produce chemicals that are of medical, technological, environmental, or scientific value, such as sugars, alcohols, proteins, antibodies, short-chain fatty acids, and other biochemicals that can be produced by appropriately engineered Saccharomyces cervisiae, Chinese hamster ovary (CHO) cells, or other mammalian, microbial, fungal, or algal cells. Increasing the efficiency with which these cells convert input nutrients to output biological products is extremely important to minimize the use of valuable input feed stocks while maximizing the amount of product produced.

In practice, several classes of bioreactors are used, including batch bioreactors, batch-fed bioreactors, continuous-perfusion bioreactors, and chemostats. In a batch bioreactor, the reactor is filled with nutrient-containing media and a small number of cells is introduced that consume nutrients, grow, and secrete the desired biological products until the nutrients are exhausted and/or the level of secreted metabolic inhibitors drive the cells to senescence or death. If the cells are adherent to the interior surfaces of the bioreactor or otherwise captured within the reactor, it is possible to replace media at a regular rate without losing the cells, i.e., by creating a intermittent (batch-fed) or continuous-perfusion bioreactor. Unless the cells are adherent to the bioreactor, captured or otherwise retained, the addition of fresh media and the removal of spent media will lead to the loss of cells, possibly at a significant economic cost. It is possible to use one of several filtering or cell separation methods to extract from the effluent stream suspended cells or cells bound to suspended carrier beads, and return them to the bioreactor, thereby maintaining a high concentration of metabolically active cells in the bioreactor to improve the economic return of the entire process.

Chemostats represent a subset of continuous-flow bioreactors used heavily to grow and study prokaryotic microbes, yeast, or suspended mammalian or other eukaryotic cells. The chemostats provide a steady supply of both fresh, cell-free, nutrient-laden media and oxygen (for cells with aerobic metabolism) that support cell growth and division within the bioreactor. An output tube is configured to limit the volume of media within the reactor, and some means for stirring and oxygenation ensures that the media within the bioreactor is well mixed and uniformly oxygenated. Once an empty bioreactor is filled to its maximum allowed volume, the constant-volume requirement implies that the steady-state rates of liquid inflow and outflow must be identical.

Because the reactor is well mixed, the liquid outflow removes from the reactor not only conditioned media but also cells. The rate at which cells are removed from the reactor is determined by both the outflow rate and the concentration of cells in that outflow. The rate at which cells are added to the bioreactor is determined by the number of cells in the bioreactor and their rate of division, which in turn is determined by the concentration of nutrients and oxygen in the reactor. As the number of cells in the reactor increases, so does their rate of consumption of nutrients and oxygen and their production of metabolites and carbon dioxide. The higher the concentration of nutrients and oxygen in the reactor, the faster the cells will be able to grow and divide, up to some biological limit; similarly, the lower the concentration of nutrients and oxygen and the higher the concentration of growth-inhibiting metabolic waste products, the slower the cells grow, where below a critical value, they either enter into senescence or die.

Upon initial seeding of the bioreactor, the number of cells in the reactor will increase until the rate of cell addition through cellular division equals the rate of their removal in the effluent, at which point the system reaches a chemical steady state, hence the name “chemostat.” Given that the cells require time, nutrients, and oxygen to grow and divide, for any particular input nutrient concentration and level of oxygen in the bioreactor there is a maximum rate at which the cells can reproduce, and hence there is a maximum input flow rate, above which all cells are eventually washed out of the reactor. If a higher concentration of cells is desired, the increased consumption of nutrients and oxygen and production of metabolic products from the growing number of cells can be offset by increasing the concentration of nutrients in the media, the rate of oxygen delivery, and the media replacement rate, up to the biological limit imposed by the minimum time a cell requires to grow and divide as compared to the residence time of the media in the reactor. For too rapid media replacement, cells will be washed out before they have the time to divide. This entire process can be described quantitatively by what is known as the Monod equation.

The monitoring of the state of the cells in a bioreactor can be readily accomplished by measurement of the pH, optical density (OD), the concentrations of dissolved oxygen and carbon dioxide, and changes in the concentration of glucose, lactate, and/or other metabolites in the bioreactor effluent as compared to the input media. All of these measurements can be accomplished non-invasively within the bioreactor or from the effluent stream. The optimization of the biomolecular production efficiency of a cell line growing in a chemostat typically requires additional, quantitative, off-line analysis of the cellular secretome, i.e., the metabolites and other biomolecules secreted by the cells during culture, as well as the cellular proteome, metabolome, lipidome, and transcriptome, which in turn can be used to guide genetic modifications of the cells to enhance biomolecular production rates or expand the range of bioreactor conditions favorable for culturing a particular cellular phenotype. The probability of identifying a cell with appropriate gene expression and determining the nutrient concentrations that are optimal for a particular production objective can be increased by examining a large number of individual clonal cell populations, which motivates the drive for creating smaller and smaller bioreactors that can be used in larger and larger numbers, e.g., by using multi-well microchemostats or other arrays of microbioreactors. While the volume of media and the number of cells required for these cellular and molecular analyses are decreasing with the introduction of advanced measurement technologies, there will always be some minimum required volume of cells and media. When cells are cultured in commercial-size bioreactors, with volumes of a thousand liters or more, the removal of a 0.5 mL sample for detailed analysis will not perturb the system, but as the volume of the bioreactor is reduced, the fractional perturbation of the withdrawal of a fixed-volume increases as shown in Table 1, which demonstrates that removing a 0.5 mL sample from a 1.0 mL chemostat would represent a 50% reduction in the volume of the media within the chemostat.

TABLE 1
Fractional change in bioreactor volume
upon removal of a 0.5 mL sample

| Reactor volume, mL | 1 | 1.5 | 15 | 250 | 1000 |
|---|---|---|---|---|---|
| Fraction withdrawn | 50% | 33% | 3% | 0.20% | 0.05% |

An obvious alternative to removing a sample from the chemostat would be to accumulate a sufficient sample from the effluent stream and then perform the analysis. The time that this would require depends upon the dilution rate, which might range from 0.01 hr⁻¹ for slowly growing CHO cells to up to ˜0.5 hr⁻¹ for the faster growing yeast Saccharomyces cervisiae, and 0.6 hr⁻¹ for bacteria such as E. coli. As shown in Table 2, the smaller the reactor, the slower the flow rate required to obtain the requisite dilution, and as shown in Table 3, the longer the time required to collect the 0.5 mL sample.

TABLE 2
Flow rate in μL/min for specified dilutions
in chemostats with differing volumes

| | | Chemostat volume, mL: | | | | |
|---|---|---|---|---|---|---|
| | | 1.0 | 1.5 | 15 | 250 | 1000 |
| Dilution rate, | 0.01 | 0.17 | 0.25 | 2.5 | 42 | 170 |
| hr⁻¹ | 0.1 | 1.7 | 2.5 | 25.0 | 420 | 1700 |
| | 0.5 | 8.3 | 13 | 130 | 2,100 | 8,300 |
| | 1 | 17.0 | 25 | 250 | 4,200 | 17,000 |

TABLE 3
Time (min) to collect 0.5 mL of effluent
for differing dilution rates and volumes

| | | Chemostat volume, mL: | | | | |
|---|---|---|---|---|---|---|
| | | 1.0 | 1.5 | 15 | 250 | 1000 |
| Dilution rate, | 0.01 | 3000 | 2000 | 200 | 12 | 0.03 |
| hr⁻¹ | 0.1 | 300 | 200 | 20 | 1.2 | 0.03 |
| | 0.5 | 60 | 38 | 3.8 | 0.24 | 0.03 |
| | 1 | 29 | 20 | 2.0 | 0.12 | 0.03 |

Tables 2 and 3 illustrate the problem with accumulating measurement-sized samples from low volume chemostats at low dilution rates: it could take many hours to accumulate the sample, for example 200 minutes to collect a 0.5 mL sample from a 1.5 mL bioreactor operating at a dilution rate of 0.1 hr⁻¹. This poses a significant problem, in that unless all biochemical activity is halted in the collected sample, biochemical reactions will continue in the container as sample is being accumulated, but the rates and products of these reactions may have no semblance of the rates and products of the reactions occurring within the chemostat because this sample is not being fed fresh media, may have a different composition of dissolved gases, and may have different temperature and stirring parameters. Simply lysing the cells upon collection will be insufficient, since biochemical reactions will continue even in cell lysate.

Therefore, a heretofore unaddressed need exists in the art to address the aforementioned deficiencies and inadequacies.

### SUMMARY OF THE INVENTION

One aspect of the present invention relates to a chemostat comprising a chamber containing media with cells for cell growth and cell division; an input tube coupled to the chamber for delivering at an inflow rate nutrient-laden media that support the cell growth and the cell division within the chamber; a tube for delivering an input gas mixture into the media; a tube for venting the gas from the chamber head space; and an output tube coupled to the chamber for withdrawing a sample of media with cells from the chamber at an outflow rate. The inflow rate and the outflow rate of the media are regulated such that the chemostat is operable in a sample accumulation phase or a sample withdrawal phase immediately following the sample accumulation phase, wherein in the sample accumulation phase, the media outflow rate is zero or very low and the media inflow rate increases in proportion to an instantaneous volume of media in the chamber so as to accumulate the sample without changes in a metabolic state within the chamber; and wherein in the sample withdrawal phase, the outflow rate is regulated at a significantly higher rate than the inflow rate to withdraw the accumulated sample from the chamber rapidly at one time. Throughout the withdrawal process, the inflow rate is adjusted to remain in proportion to the instantaneous volume, thereby maintaining a steady chemical state, termed chemostasis, in the chamber.

In one embodiment, the chemostat is a variable-volume chemostat in which the instantaneous volume of media varies with time.

In one embodiment, the quantity of nutrients per cell within the media in the chamber remains unchanged.

In one embodiment, the chemostat is configured such that a dilution rate that is a ratio of the inflow rate divided by the instantaneous volume is a constant, so as to maintain the same ratio of nutrient delivery per cell independent of the total volume of the media and the cells it contains.

In one embodiment, a maximum volume of the sample that is accumulated and then removed is determined by a difference between a maximum allowable volume and a minimum allowable volume of the chamber, and the time required to accumulate the sample is determined by the maximum volume of the sample divided by the inflow rate.

In one embodiment, the inflow rate and the outflow rate are adjustable simultaneously to support different phases of sample accumulation and sample withdrawal.

In one embodiment, the inflow rate and the outflow rates are of different functions of time such that a difference between the inflow rate and the outflow rate equals a rate of change of the instantaneous volume with time.

In one embodiment, when the inflow rate is greater than the outflow rate, then the volume of fluid within the chemostat increases with time, and when the outflow rate is greater than the inflow rate, the volume of fluid decreases in time.

In one embodiment, the chemostat is operable in a filling phase during which the outflow rate is zero and the inflow rate is greater than zero so that the instantaneous volume increases with time, or in a steady-state phase during which the outflow rate is same as the inflow rate.

In one embodiment, the chemostat further comprises an output pump coupled to the output tube for regulating the outflow rate, and an input pump coupled to the input tube for regulating the inflow rate.

In one embodiment, the output pump is turned off at the beginning of the sample accumulation phase and turned on at the beginning of the sample withdrawal phase.

In one embodiment, the chemostat further comprises a means for stirring and oxygenating the media in the chamber respectfully at a stirring rate and a gas exchange rate to ensure that the media within the chamber is well mixed, uniformly oxygenated, and at the desired pH over the full range of volumes of media contained in the bioreactor chamber during all phases of operation.

In one embodiment, the input gas mixture composition, the gas exchange rate, and the stirring rate are adjustable so as to ensure that the local conditions throughout the bioreactor chamber remain unchanged over the full range of volumes of media contained in the bioreactor chamber during all phases of operation.

In one embodiment, growth conditions and nutrient and gas concentrations within the entire media in the chamber are maintained at original conditions by modulating the inflow rate, the outflow rate, the gas exchange rates, and the stirring rate in a manner that maintains static biochemical conditions independent of the instantaneous volume of media and cells within the chamber.

In one embodiment, a fraction of the media in the chamber is withdrawable without prior accumulation, but with the inflow rate, the outflow rate, the gas exchange rate, and the stirring rate modulated post-withdrawal in a manner that maintains static biochemical conditions independent of the instantaneous volume of cells and media within the chamber.

In one embodiment, by the dynamic control of the inflow rate, the outflow rate, the gas exchange rate, and the stirring rate, any arbitrary volume within the chamber can be maintained at the same biochemical state as any volume within an industry-standard, constant-volume chemostat.

In another aspect, the invention relates to a method for operating a chemostat. The chemostat is characterized with a media volume that varies with time in a chamber, an inflow rate at which nutrient-laden media are delivered into the chemostat, and an outflow rate at which a sample is withdrawn from the chemostat.

The method comprises regulating the inflow rate, the outflow rate, and the input gas mixture such that the chemostat operates in a sample accumulation phase or a sample withdrawal phase immediately following the sample accumulation phase, wherein in the sample accumulation phase, the outflow rate is zero or very small and the inflow rate and gas exchange rate increase in proportion to an instantaneous volume of media in the chamber so as to accumulate the sample without changes in a metabolic state within the chamber; and wherein in the sample withdrawal phase, the outflow rate is regulated at a higher rate than the inflow rate to withdraw the accumulated sample from the chamber rapidly at one time and the inflow rate and gas exchange rate remain in proportion to the instantaneous volume to maintain chemostasis in the chamber.

In one embodiment, a dilution rate that is a ratio of the inflow rate divided by the instantaneous volume is a constant, so as to maintain the same ratio of nutrient delivery per cell independent of the total volume of the media and cells.

In one embodiment, the method further comprises stirring and oxygenating the media in the chamber respectfully at a stirring rate and a gas exchange rate to ensure that the media within the chamber is well mixed, uniformly oxygenated, and has the desired carbon dioxide levels.

In one embodiment, the gas exchange rate and the stirring rate are adjustable so as to ensure that the local conditions throughout the bioreactor chamber remain unchanged.

These and other aspects of the invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment.

FIG. 1 shows schematically a yeast chemostat with input and output pumps, gas mixture delivery, gas venting, and a stirring means according to embodiments of the invention. In operation as a classical chemostat, the input and output flow rates are equal so that the volume is constant over time. Were this a variable-volume chemostat, the instantaneous input flow rate would be determined by the volume at each moment of time.

FIGS. 2A-2C show schematic representations of three time points in the operation of a variable-volume chemostat. FIG. 2A: Steady-state phase; FIG. 2B: End of sample accumulation phase; and FIG. 2C: End of withdrawal phase. Note that the media and gas flow rates in FIG. 2B are twice those in FIG. 2A, as would be appropriate with the media volume in FIG. 2B twice that in FIG. 2A. In FIG. 2C, the output flow is transiently very high as the input and gas flows return to their baseline values, consistent with bioreactor volume approaching the steady-state value.

FIG. 3 shows the time-sequence of sample accumulation and withdrawal in a variable-volume chemostat according to embodiments of the invention. A) Filling chemostat phase: No withdrawal, volume increases linearly with time. B) Steady-state phase: delivery=withdrawal. C) Sample accumulation phase: No withdrawal, delivery rate increases in proportion to volume to maintain chemical steady state, with slight exponential curvature evident in Volume. D) Sample withdrawal phase: accelerated sample withdrawal, media delivery remains in proportion to volume to maintain chemostasis, with an almost undetectable curvature. The gas mixture would be regulated as necessary during all phases.

### DETAILED DESCRIPTION OF THE INVENTION

The invention will now be described more fully hereinafter with reference to the accompanying drawings, in which exemplary embodiments of the invention are shown. The invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the invention or of any exemplified term. Likewise, the invention is not limited to various embodiments given in this specification.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the invention.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

It will be understood that the terms “gas delivery” and “gas and pH regulation” will comprise the regulatory steps required to maintain constant concentrations of dissolved oxygen and carbon dioxide and a constant pH in the media as the volume of media and total number of cells in the bioreactor are changed. In all discussions of the operation of a chemostat in variable volume mode, it will be assumed in every instance that gas and pH regulation can be applied whenever appropriate, and hereafter will not be addressed specifically.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which the invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “around,” “about,” “substantially” or “approximately” shall generally mean within 20 percent, preferably within 10 percent, and more preferably within 5 percent of a given value or range. Numerical quantities given herein are approximate, meaning that the terms “around,” “about,” “substantially” or “approximately” can be inferred if not expressly stated.

As used herein, the terms “comprise” or “comprising,” “include” or “including,” “carry” or “carrying,” “has/have” or “having,” “contain” or “containing,” “involve” or “involving” and the like are to be understood to be open-ended, i.e., to mean including but not limited to.

As used herein, the phrase “at least one of A, B, and C” should be construed to mean a logical (A or B or C), using a non-exclusive logical OR. As used herein, the term “and/or” includes any and all combinations of one or more of the associated listed items.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

Chemostats are a type of continuous-flow bioreactors used to grow and study prokaryotic microbes such as E. coli, yeast, or suspended mammalian or other eukaryotic cells grown in suspension. This invention provides a novel approach to remove samples from a chemostat in a manner that does not disrupt the biochemical equilibrium within the chemostat. In a conventional chemostat, a continuous flow of media into the bioreactor is matched by an equal and opposite flow of conditioned media and cells out of the bioreactor, so that the volume of fluid in the bioreactor is a constant. As cells grow and divide, some of the cells are washed out. Unless the flow rates are so high as to wash all the cells from the bioreactor, the system will reach a steady state determined by both the flow rate and the concentration of nutrients in the input stream, and the chemical conditions and number of cells in the chemostat at any time will remain constant, hence the term “chemostat,” with “chemo” referring to the biochemistry within the reactor and “stat” referring to “static.” The effluent from a chemostat can be collected for analysis or transfer to another bioreactor. As the chemostats are made smaller, the flow rate required to maintain the smaller number of cells is reduced correspondingly, thereby extending the time required to collect a specific volume for analysis. However, the biochemical processes occurring within the bioreactor might continue in the sample collection portion of the apparatus, which has different growth conditions and nutrient and gas concentrations. The ongoing biochemistry in the collection portion of the apparatus could be slowed or halted by immediately freezing the sample or heating it to a point where enzymes are degraded, both of which complicate the sample collection process, particularly when it is desired to operate a large number of chemostats in parallel. A similar problem occurs when a significant fraction of the volume of a chemostat is needed to inoculate another bioreactor, in that removal of a fraction of the media and cells from a chemostat could lead to altered biochemical conditions for the remaining cells, which might be needed to grow more cells to seed additional bioreactors.

There are two well-known solutions to this problem: stop protein biochemistry by flash heating the sample as it is being collected, with or without lysing, to denature proteins, or freeze it to approximately −80° C. The limitations of the former are that not all biochemical reactions within cell media or lysate are driven by enzymes or other proteins and will continue even after protein denaturation, and it may be difficult to properly denature proteins without producing peptide fragments or altering other biomolecules. The limitations of the latter are that biochemistry will resume as the sample is being thawed for analysis, and it may be difficult to simultaneously thaw the entire sample. In either case, the problems in making biochemical measurements on samples that are collected over time will limit the ability to quantify short-lived or labile species in the sample.

In view of the aforementioned deficiencies and inadequacies, this invention provides a novel approach: the media (fluid) in the chemostat is allowed to accumulate in the chemostat portion of the apparatus by slowing or stopping the media removal rate. The excess volume constitutes the sample that is to be removed from the chemostat rapidly at one time. The growth conditions and nutrient and gas concentrations within the entire volume in the chemostat are maintained at the original conditions by modulating the input flow (or inflow) rate, the output flow (or outflow) rate, the gas exchange rates, and the stirring rate in a manner that maintains static biochemical conditions independent of the instantaneous volume of cells and media within the chemostat chamber. Similarly, a substantial fraction of a chemostat could be removed without prior accumulation, but with the input flow, output flow, gas exchange, and stirring rates modulated post-withdrawal in a manner that maintains static biochemical conditions independent of the instantaneous volume of cells and media within the chemostat chamber. The variable volume phase of chemostat operation could occur before or after sample removal, depending upon whether the chemostat had any available excess volume or could support growth from a partially filled chemostat. The input and output flow rates can both be modulated simultaneously to support different durations of sample removal and volume recovery. Hence the dynamic control of bioreactor parameters such as the rates of input flow (or inflow), output flow (or outflow), gas exchange, and stirring can hold any arbitrary volume within the chemostat chamber at the same biochemical state as any volume within an industry-standard, constant-volume chemostat, as would be required to harvest a substantial fraction of a chemostat's volume for either sample analysis or the seeding of another bioreactor.

While one might view a “variable-volume chemostat” as an oxymoron, it is important to realize that the key property of a conventional chemostat is that it holds all of the cells it contains in a biochemical steady state, hence the name. The uniform biochemical state of a conventional, well-mixed chemostat implies that each incremental volume element within that chemostat is in the same steady chemical state. Just as density is an intrinsic variable describing mass per unit volume and the mass of an object is an extrinsic variable representing the integral of the density over the entire volume of an object, we make the distinction of an “intrinsic chemostasis” and “extrinsic chemostasis.” Within a variable-volume chemostat, each elemental volume enjoys an identical intrinsic chemostasis, but the chemostat in its entirety is not static since its volume and the number of elemental volumes within it changes over time. Hence it is appropriate to describe a variable-volume chemostat as an intrinsic chemostat. In contrast, a classical, fixed-volume chemostat exhibits both intrinsic and extrinsic chemostasis.

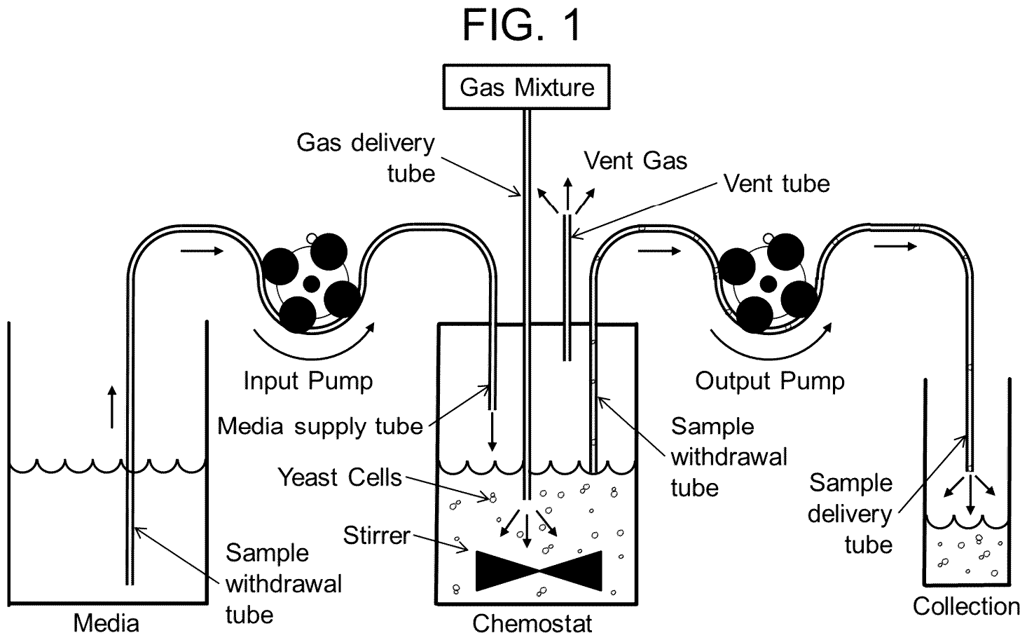

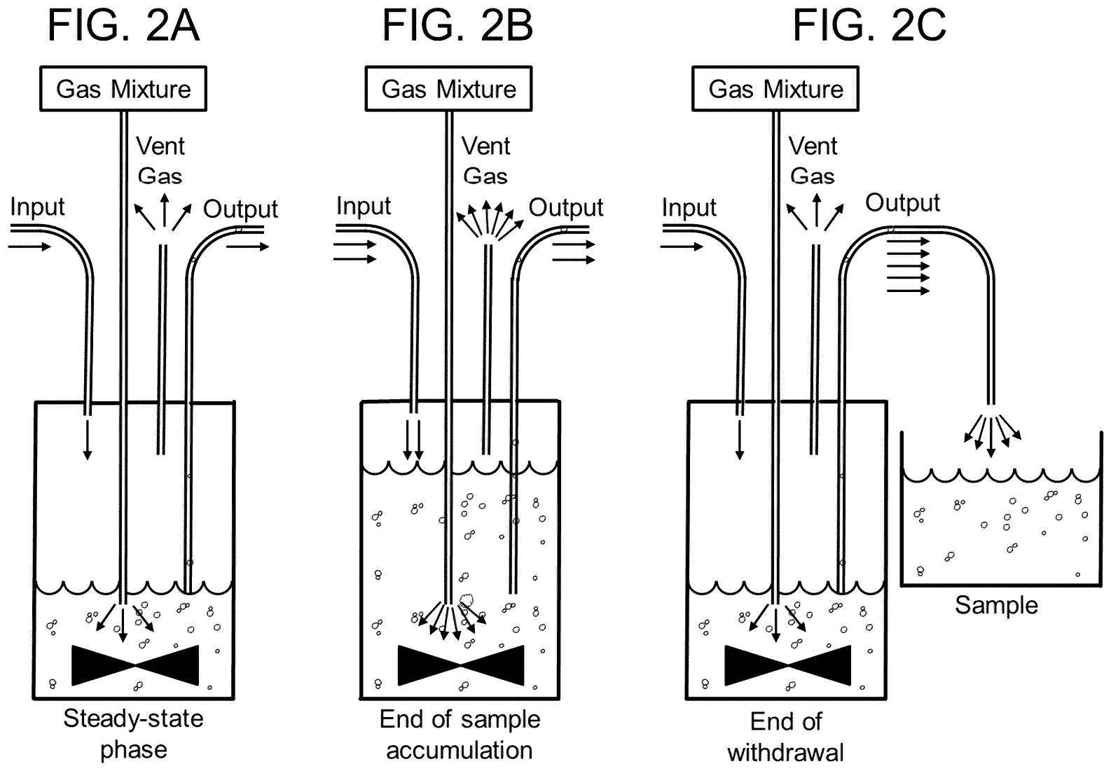

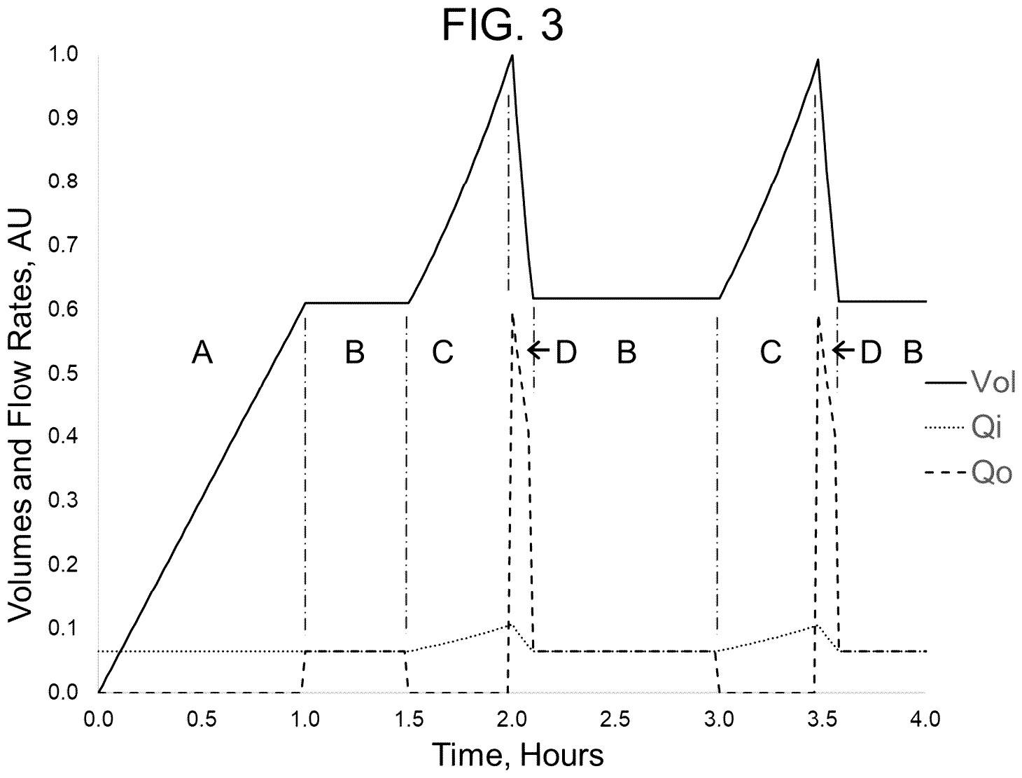

Referring to FIG. 1, the chemostat comprises a chamber containing media with cells for cell growth and cell division; an input tube coupled to the chamber for delivering nutrient-laden media that support the cell growth and the cell division within the chamber, at an inflow rate; and an output tube coupled to the chamber for withdrawing a sample from the chamber at an outflow rate. As illustrated schematically in FIGS. 2A-2C and temporally in FIG. 3, the inflow rate and the outflow rate are regulated such that the chemostat is operable in a sample accumulation phase or a sample withdrawal phase immediately following the sample accumulation phase. In the sample accumulation phase, the outflow rate is zero and the inflow rate increases in proportion to an instantaneous volume of media in the chamber so as to accumulate the sample without changes in a metabolic state within the chamber. In the sample withdrawal phase, the outflow rate is regulated at a higher rate than the inflow rate to withdraw the accumulated sample from the chamber rapidly at one time and the inflow rate remains in proportion to the instantaneous volume to maintain chemostasis in the chamber. As discussed previously, depending on the operational protocol, gas and pH may need to be regulated during these phases.

In some embodiments, the chemostat is a variable-volume chemostat in which the instantaneous volume of media varies with time.

In some embodiments, the quantity of nutrients per cell within the media in the chamber remains unchanged in different phases of sample accumulation and sample withdrawal.

In some embodiments, the chemostat is configured such that a dilution rate that is a ratio of the inflow rate divided by the instantaneous volume is a constant, so as to maintain the same ratio of nutrient delivery per cell independent of the total volume of the media and the cells it contains.

In some embodiments, a maximum volume of the sample that is accumulated and then removed is determined by a difference between a maximum allowable volume and a minimum allowable volume of the chamber, and the time required to accumulate the sample is determined by the maximum volume of the sample divided by the inflow rate.

In some embodiments, the inflow rate and the outflow rate are adjustable simultaneously to support different phases of sample accumulation and sample withdrawal.

In some embodiments, the inflow rate and the outflow rate are of different functions of time such that a difference between the inflow rate and the outflow rate equals a rate of change of the instantaneous volume with time.

In some embodiments, when the inflow rate is greater than the outflow rate, then the volume of fluid within the chemostat increases with time, and when the outflow rate is greater than the inflow rate, the volume of fluid decreases in time.

In some embodiments, the chemostat is operable in a filling phase during which the outflow rate is zero and the inflow rate is greater than zero so that the instantaneous volume increases with time, or in steady-state phase during which the outflow rate is same as the inflow rate.

In some embodiments, the chemostat further comprises an output pump coupled to the output tube for regulating the outflow rate, and an input pump coupled to the input tube for regulating the inflow rate.

In some embodiments, the output pump is turned off at the beginning of the sample accumulation phase and turned on at the beginning of the sample withdrawal phase.

In some embodiments, the chemostat further comprises a means for stirring and oxygenating the media in the chamber respectfully at a stirring rate and a gas exchange rate to ensure that the media within the chamber is well mixed, uniformly oxygenated, and at the desired pH over the full range of volumes of media contained in the bioreactor chamber during all phases of operation.

In some embodiments, the input gas mixture composition, the gas exchange rate and the stirring rate are adjustable so as to ensure that the local conditions throughout the bioreactor chamber remain unchanged over the full range of volumes of media contained in the bioreactor chamber during all phases of operation.

In some embodiments, growth conditions, and nutrient and gas concentrations within the entire media in the chamber are maintained at original conditions by modulating the inflow rate, the outflow rate, the gas exchange rates, and the stirring rate in a manner that maintains static biochemical conditions independent of the instantaneous volume of media and cells within the chamber.

In some embodiments, a fraction of the media in the chamber is withdrawable without prior accumulation, but with the inflow rate, the outflow rate, the gas exchange rate, and the stirring rate modulated post-withdrawal in a manner that maintains static biochemical conditions independent of the instantaneous volume of cells and media within the chamber.

In some embodiments, by the dynamic control of the inflow rate, the outflow rate, the gas exchange rate and the stirring rate, any arbitrary volume within the chamber can be maintained at the same biochemical state as any volume within an industry-standard, constant-volume chemostat.

In another aspect, the invention relates to a method for operating a chemostat. The chemostat is characterized with a media volume that varies with time in a chamber, an inflow rate at which nutrient-laden media are delivered into the chemostat, and an outflow rate at which a sample is withdrawn from the chemostat.

The method comprises regulating the inflow rate, the outflow rate, and the input gas mixture such that the chemostat operates in a sample accumulation phase or a sample withdrawal phase immediately following the sample accumulation phase, wherein in the sample accumulation phase, the outflow rate is zero or very small and the inflow rate and the gas exchange rate increase in proportion to an instantaneous volume of media in the chamber so as to accumulate the sample without changes in a metabolic state within the chamber; and wherein in the sample withdrawal phase, the outflow rate is regulated at a higher rate than the inflow rate to withdraw the accumulated sample from the chamber rapidly at one time and the inflow rate and the gas exchange rate remain in proportion to the instantaneous volume to maintain chemostasis in the chamber.

In one embodiment, a dilution rate that is a ratio of the inflow rate divided by the instantaneous volume is a constant, so as to maintain the same ratio of nutrient delivery per cell independent of the total volume of the media and cells.

In one embodiment, the method further comprises stirring and oxygenating the media in the chamber respectfully at a stirring rate and a gas exchange rate to ensure that the media within the chamber is well mixed and uniformly oxygenated, and has the desired carbon dioxide levels.

In one embodiment, the gas exchange rate and the stirring rate are adjustable so as to ensure that the local conditions throughout the bioreactor chamber remain unchanged.

In addition, in some embodiments, whether the chemostat is being operated in an aerobic or anaerobic mode and the values of other cell culture parameters would determine whether it is necessary to adjust over time buffer concentrations in the input media and the partial pressures of oxygen, carbon dioxide, and nitrogen in the incoming gas mixtures to maintain constant concentrations of dissolved oxygen and carbon dioxide and a constant pH in the media as the volume of media and total number of cells in the bioreactor are changed. In some embodiments, this process is referred to as “gas and pH regulation.

In some embodiments, the method may also include the regulatory steps for maintaining constant concentrations of dissolved oxygen and carbon dioxide and a constant pH in the media as the volume of media and total number of cells in the bioreactor are changed. In all discussions of the operation of a chemostat in variable volume mode, it is assumed in every instance that gas and pH regulation can be applied whenever appropriate, and hereafter will not be addressed specifically.

These and other aspects of the invention are further described below.

As disclosed above, the novel approach of the invention is not to move the sample to a separate container for accumulation and subsequent analysis, but to store the sample within the chemostat where the entire sample and the other cells in the chemostat will enjoy the same conditions while the sample is being accumulated. However, it is the unquestioned dogma that chemostats have constant volume, achieved by having equal inflow and outflow rates. Relaxing the constant volume constraint in fact solves the accumulation problem, as long as the local conditions within the chemostat are unchanged as its volume increases. Suppose that the output pump is turned off at the beginning of the sample accumulation period, so that the volume increases linearly with time, as determined by the input flow rate. During this time, the number of cells in the chemostat will increase, since cells are no longer being removed. There will be an increase in the total amount of metabolites in the chemostat, but because new media is being added at a rate proportional to the bioreactor volume, the concentration of these metabolites will remain constant if the cell metabolic rates are unchanged and the media inflow remains constant. However, if the inflow rate is held constant during sample accumulation, the increase in chemostat volume will mean that the nutrients being delivered will be shared by the ever-growing number of cells, for a net decrease in available nutrients per cell. This in turn will reduce the rate at which metabolites are produced. This means that simply turning off the output pump is an inadequate means for accumulating a sample that is also representative of the steady-state phase that preceded sample accumulation.

Hence under a variable-volume protocol, the flow rate must be increased in proportion to the increase in volume so that the nutrients available per cell will remain unchanged. Appropriate changes in gas exchange and stirring will ensure that the local conditions throughout the bioreactor remain unchanged. In the standard, constant-volume chemostat, the effluent immediately after its removal is only initially in equilibrium with the bulk media in the chemostat, and that equilibrium is quickly lost as the metabolically active cells are confined to the removal tubing or a passive collection reservoir where fresh nutrients are no longer being delivered to cells, gas is no longer being exchanged, and the only stirring in the tube comes from the shear force applied by viscous drag against the tubing wall, and the collection reservoir may not be stirred. In the variable-volume chemostat, the accumulated sample remains in equilibrium within the chemostat, with the same nutrient delivery rate per cell, the same gas exchange, and the same stirring forces. In other words, the variable volume chemostat stores the accumulating sample within itself. While this feature is not needed for large chemostats, where the removal of a sample does not appreciately affect the remaining chemostat volume. In contrast, the removal of a small sample from an only slightly larger classic chemostat would be metabolically disruptive to chemostat.

FIGS. 2A-2C show schematic representations of three time points in the operation of a variable-volume chemostat. FIG. 2A shows the steady-state phase, where the delivery equals the withdrawal, and the volume is constant. FIG. 2B represents the chemostat near the end of sample withdrawal, where the media and gas flow rates are still twice those in FIG. 2A, as would be appropriate with the media volume in FIG. 2B being twice that in FIG. 2A. FIG. 2C shows the end of sample accumulation, where the output flow is transiently very high while the input and gas flows are returning to their baseline values, consistent with bioreactor volume approaching the steady-state value. The gas mixture would be regulated as necessary during all stages of sample accumulation and delivery. It is important to recognize that at the three instances shown, the metabolic states of all the cells shown is identical, even those in the just-removed sample, hence the value of the variable-volume chemostat.

FIG. 3 shows schematically the time sequence of sample accumulation and withdrawal in the variable-volume chemostat. There is a need to accumulate sample for periodic removal, and off-line analysis could affect chemostasis. The solution is to change the input pump rate, so that when the output pump is OFF to accumulate the sample, the delivery pump flow rate is increased so that the media delivery rate is always in proportion to the volume of media and cells in the chemostat. Phase A) Filling chemostat: no withdrawal, and volume increases with time. Phase B) Steady state: delivery=withdrawal. Phase C) Sample accumulation: No withdrawal, and delivery rate increases in proportion to volume to maintain chemical steady state. Phase D) Sample withdrawal: Accelerated sample withdrawal, and media delivery remains in proportion to volume to maintain chemostasis. It should be noted that the transition between phases A-D would be smooth, but any discontinuous derivatives evident in the graph reflect the discretization of the equations used to generate the traces, not the underlying hydrodynamics.

It is possible, but not shown in FIG. 3, that during the sample collection phase the output flow rate is not zero but instead kept very small to ensure that there are no cells trapped in the output tube during what could be an extended sample accumulation phase, since these cells would still be alive but increasingly deprived of appropriate access nutrients and media.

As shown in FIG. 3, after filling, a chemostat is operated at a constant, initial media delivery rate, Qᵢ, that is determined by the bioreactor volume and the desired dilution rate, and the media removal flow rate, Qₒ, is matched to the input delivery rate.

At time Tₛ, the removal pump is turned off, and the instantaneous delivery rate is determined by the instantaneous volume, V, to produce the same system dilution rate. The rates of gas exchange and stirring are increased accordingly.

There are a number of different practical considerations in operating a variable-volume chemostat. In a classic chemostat, the dilution rate, D, is the ratio of the constant input flow rate Qⁱⁿ divided by the constant volume of the chemostat, V, such that

D=Qⁱⁿ/V,

where D has units of inverse time, typically inverse hours. The reciprocal of D is the time required to fill an empty chemostat with the flow rate Qⁱⁿ.

In the variable-volume chemostat, the maximum volume of a sample that can be accumulated and then removed is determined by the difference between the maximum and minimum allowable volumes of the bioreactor,

Vₛₐₘₚₗₑ=Vₘₐₓ−Vₘᵢₙ,

and the time required to accumulate the sample Tₛₐₘₚₗₑ is simply

Tₛₐₘₚₗₑ₌Vₛₐₘₚₗₑ/Qⁱⁿ.

Under the variable volume paradigm, the instantaneous inflow rate, Qⁱⁿ(t), is proportional to the instantaneous volume of the bioreactor V(t), where the constant of proportionality is simply the dilution rate, D, which is assumed to be constant so as to maintain the same ratio of nutrient delivery per cell independent of the total volume of the cells, such that

Qⁱⁿ(t)=D×V(t).

This means that the input flow rate per unit volume is a constant

Qⁱⁿ(t)/V(t)=D,

In a constant volume chemostat, the input and output flow rates are equal, i.e.,

Qᵢₙ=Q^{out},

so that Qⁱⁿ−Q^{out}=0, and the volume remains constant.

In the variable-volume chemostat, the input and output flow rates can be different functions of time, such that their difference equals the rate of change of volume of the chemostat, i.e.,

Qⁱⁿ(t)−Q^{out}(t)=dV(t)/dt.

Hence, if the input flow rate is greater than the output flow rate, then the volume of fluid within the chemostat increases with time. If the output flow rate is greater than the input flow rate, the volume of fluid decreases in time, as illustrated in FIG. 3.

Referring to FIG. 1, which shows one embodiment of the chemostat, we see that in steady-state operation, the output pump is set at a pumping rate greater than the input pump rate, i.e., output overpumping.

The maximum bioreactor volume is determined by the height of the longer tube on the right side that protrudes deeper into the chemostat reservoir. The shorter tube on the left is designed not to come in contact with the media to avoid back contamination. Starting with an empty chemostat, the fluid level rises at a rate determined by the input pump rate. Once the media rises to make contact with the withdrawal tube, the output pump withdraws media. The solution is drawn until the meniscus falls below the tube on the right and breaks, allowing the output pump to pump air into the output tube rather than water. The process then repeats, refilling the chemostat until fluid is again withdrawn. The extraction volume of each cycle is dependent upon surface tension and contact angle of the tubes, both of which could be adjusted to affect the volume of the extracted bolus of media. Either continuous or pulsatile stirring can also affect the frequency and volume of the fluid withdrawals, as could the cross-sectional shape of the chemostat reservoir. The spacing of bubbles in the output line will reflect the frequency with which the meniscus is made and broken.

In a practical embodiment of a variable-volume chemostat, as might be implemented with the design shown in FIG. 1, the output pump is periodically turned off so that the fluid level rises inside the chemostat. The input pumping rate is increased in proportion to the instantaneous volume of the chemostat until the desired volume has been accumulated, at which time the output pump is turned on at a significantly higher rate than the input pump to quickly deliver the accumulated sample to the output tube and downstream sensors or collection devices. As this withdrawal is occurring, the input flow rate has to be decreased accordingly, as shown in FIG. 3. The net result is that samples of cells and media are accumulated within the chemostat, where they maintain the same conditions as the rest of fluid and cells in the chemostat. Their rapid removal at the end of a possibly lengthy accumulation interval ensures that the biochemistry can be quickly and uniformly stopped during or immediately after the sample removal process.

As a final point, in order to better appreciate the difference between a fixed volume and a variable-volume chemostat, it is useful to revisit the difference between intensive and extensive variables. As a second example, the heat capacity of an object is an extensive variable whose value depends upon the mass of the object. The specific heat capacity, or specific heat, is the heat capacity divided by the mass and hence is an intrinsic variable determined by the thermal properties of the material and not the amount of material present. Dilution rate, D, is an intrinsic variable, while a constant input flow rate, Qⁱⁿ, is an extrinsic variable that will need to be large for a large bioreactor and small for a small one. In effect, we have created an intrinsic system variable Qⁱⁿ(t)/V(t), which could be termed a “specific flow rate.” As long as oxygenation and stirring are scaled similarly, the creation of specific or intrinsic variables is what allows the chemostat to have a variable volume. However, it is important to recognize that the density of the chemostat effluent that contains cells, reduced nutrients, and added metabolites may be higher than the density of the input media, which is free of cells and metabolites. In this context, it is important to recognize the difference between mass flow rates and volumetric flow rates. In a classic chemostat, the input mass flow rate must be the same as the output mass flow rate, since mass is conserved in the chemistry, and otherwise the mass of the chemostat would be changing with time. However, if the input density of the input medium is less than the density of the output medium, the volume flow rate at the input will be higher than the volume flow rate at the output. These effects are small, but it may be worthwhile to consider them in the operation of a variable-volume chemostat.

In a classic chemostat, a peristaltic pump delivers input media at a known volumetric flow rate, and a siphon that is part of the fixed volume bioreactor maintains the constant volume within the chemostat. The volumetric output flow rate need not be measured because it is set by the input flow rate and the constant volume; were there a change in density between inflow and outflow, it might not be noticed and with this regulatory method would be irrelevant. Were volumetric pumps used on both the input and the output, flow rates would have to be adjusted to ensure that the volume remained constant.

The variable-volume chemostat has a volume that can change in time, wherein the input and output flow rates are adjusted such that the biomass density and the density flow rates in the variable-volume chemostat are identical to those in the classical chemostat. The biomass density and the density flow rates are intrinsic variables, in that any changes in the volume V(t) of the bioreactor are balanced by the corresponding changes in the flow rates Q(t). Hence, if the classic and variable-volume chemostats have the same intrinsic variables, they have to be biochemically indistinguishable. The variable-volume chemostat has the advantage of providing a means to accumulate a sample without allowing any changes in metabolic state during accumulation.

The same principles can be applied to create a variable volume turbidistat, wherein the specific optical density of the cells and medium are held constant independent of the volume of fluid contained in the system.

Without intent to limit the scope of the invention, further details of a classical constant volume chemostat and novel variable-volume chemostats according to the embodiments of the invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### The Classical Constant Volume Chemostat

A chemostat bioreactor is a continuously stirred tank reactor (CSTR) for which the volumetric inflow rate of input media, Qⁱⁿ, is the same as the volumetric outflow rate of the bioreactor's media, Q^{out}, and the volume of the bioreactor, V, is constant over time. Due to chemostats being well-stirred, the outflow media concentrations and biomass concentration is assumed to be the same as the reactor media concentrations, cⱼ where j=1, 2, . . . , and the reactor biomass concentration, c_{biomass}. The inflow media concentrations, cⱼⁱⁿ where j=1, 2, . . . , typically only contains essential nutrients and one or more substrates, such as glucose. Biomass is not usually fed into the chemostat so c_{biomass}ⁱⁿ=0. The differential equation for a CSTR is given as

$$\frac{d [ {c}_{j} V ]}{dt} = {Q}^{i n} {c}_{j}^{i n} - {Q}^{out} {c}_{j} + {R}_{j} V ,$$

where Rⱼ is the net reaction rate. In the case of constant volume, this equation reduces to

$$\frac{{dc}_{j}}{dt} = \frac{{Q}^{i n}}{V} {c}_{j}^{i n} - \frac{{Q}^{o u t}}{V} {c}_{j} + {R}_{j} .$$

At a steady state, the biochemistry within the chemostat does not change with time. Substrate-limited chemostats can control the stable steady states of the reactor's media concentrations by changing the inflow concentration of substrates. In this case, the physical density of cells in the media or the optical density of the cell-containing media will reflect the extent to which limiting the supply of a particular substrate in the media affects the cell's metabolic activity, allowing the determination of how growth rate is affected by the concentration of a particular substrate whose concentration is reduced below “normal” levels.

The production rate of biomass is referred to as the growth rate, μ. The growth rate is heavily dependent on substrate concentration. The steady-state growth rate, μₛₛ, in a substrate-limited chemostat is equal to the volumetric outflow rate divided by the reactor volume. This ratio,

$$\frac{{Q}^{o u t}}{V} ,$$

is referred to as the dilution rate. If the dilution rate is greater than the maximum growth rate, then washout occurs where the biomass approaches zero. The differential equation describing biomass concentration overtime is

$$\frac{{dc}_{b i o m a s s}}{dt} = ( μ - \frac{{Q}_{o u t}}{V} ) {c}_{b i o m a s s} .$$

To summarize, a chemostat is a CSTR such that

$${Q}^{i n} = {Q}^{out} and \frac{dV}{dt} = 0 .$$

At the steady-state,

$$\frac{{dc}_{j}}{dt} = 0 , ∀ j ,$$

$$\frac{{dc}_{b i o m a s s}}{dt} = 0 ,$$

$${μ}_{ss} = \frac{{Q}^{o u t}}{V} if \frac{{Q}^{o u t}}{V} \leq max μ , and$$

$${μ}_{s s} = 0 if \frac{{Q}^{o u t}}{V} \geq max μ .$$

### Variable-Volume Chemostat With Approximated Volume Differential Equation

Chemostats can have heterogeneous processes, such as Saccharomyces cerevisiae producing gaseous rather than dissolved carbon dioxide from aqueous glucose during ethanol fermentation. However, typically chemostats are treated as liquid-phase systems with water as the solvent. For liquid-phase reactors with excess solvent, the differential equation for reactor volume is approximated as

$$\frac{dV}{dt} = {Q}^{i n} - {Q}^{out} .$$

This equation can be used to derive a more generalized treatment of the substrate-limited chemostat to allow for variable volume and let Qⁱⁿ not necessarily be equal to Q^{out}. Using previous equations, it can be shown that

$$\frac{d [ {Vc}_{biomass} ]}{dt} = ( μ V - {Q}^{out} ) {c}_{biomass}$$

$$\to {c}_{biomass} \frac{dV}{dt} + V \frac{{dc}_{biomass}}{dt} = ( μ V - {Q}^{out} ) {c}_{biomass}$$

$$\to {c}_{biomass} ( {Q}^{i n} - {Q}^{out} ) + V \frac{{dc}_{biomass}}{dt} = ( μ V - {Q}^{out} ) {c}_{biomass}$$

$$\to V \frac{{dc}_{biomass}}{dt} = ( μ V - {Q}^{i n} V ) {c}_{biomass}$$

$$\to \frac{{dc}_{b i o m a s s}}{dt} = ( μ - \frac{{Q}^{out}}{V} ) {c}_{biomass} .$$

This result differs from the original differential equation for c_{biomass} since the former depends on Q^{out} while the latter depends on Qⁱⁿ. The term “dilution rate” is more difficult to use in this case, since

$$\frac{{Q}^{i n}}{V}$$

is not necessarily equal to

$$\frac{{Q}^{o u t}}{V} .$$

For this reason, the rest of this discussion will not use the term “dilution rate”. The steady state growth rate for the variable-volume case is given as

$${μ}_{s s} = \frac{{Q}^{i n}}{V} if \frac{{Q}^{i n}}{V} \leq max μ , and {μ}_{s s} = 0 if \frac{{Q}^{i n}}{V} \geq max μ .$$

With this new model, we can consider a case where the bioreactor is at steady-state, and we want to increase the volume while keeping

$$\frac{{dc}_{biomass}}{df} = 0 .$$

This can be achieved as long as Qⁱⁿ=μV

### Variable-Volume Chemostat With Constant Mass Densities

Qⁱⁿ=μV only holds if we assume the volume differential equation has the form of

$$\frac{dV}{df} = {Q}^{i n} - {Q}^{out} .$$

The more generalized form of the volume differential equation is given as

$$\frac{d [ ρ V ]}{df} = {Q}^{i n} {ρ}^{i n} - {Q}^{out} ρ ,$$

where ρ=c_{biomass}M_{biomass}+ΣⱼcⱼMⱼ is the mass density of all the components of the reactor with M_{biomass} and Mⱼ being molecular weights, and ρⁱⁿ=c_{biomass}ⁱⁿM_{biomass}+ΣⱼcⱼⁱⁿMⱼ is the mass density of all the components of the input media. In the case that the total mass density is constant, ρ=ρⁱⁿ, this equation reduces to

$$\frac{dV}{df} = {Q}^{i n} - {Q}^{out} .$$

### Variable-Volume Chemostat With Non-Constant Mass Densities

For non-constant densities, ρ≠ρⁱⁿ, it is useful to utilize an equation of state, f(c_{biomass}, c₁, c₂, . . . )=0, to derive a new differential equation for volume. Equations of state relate the thermodynamic properties (temperature, pressure, volume, composition, etc.) to each other.

1=c_{biomass}V̅_{biomass}+ΣⱼcⱼV̅ⱼ,

where V̅_{biomass} and V̅ⱼ are partial molar volumes.

The equation of state can be used to express the volume differential equation as

$$\frac{dV}{dt} = \frac{{Q}^{i n}}{ϕ} ( {f}_{biomass} {c}_{biomass}^{i n} + {\sum }_{j} {f}_{j} {c}_{j}^{i n} ) - {Q}^{out} + \frac{V}{ϕ} ( {f}_{biomass} μ + {\sum }_{j} {f}_{j} {R}_{j} ) ,$$

$$where$$

$$ϕ = {f}_{biomass} {c}_{b i o m a s s} + {\sum }_{j} {f}_{j} {c}_{j} , {f}_{j} = \frac{\partial f}{\partial {c}_{j}} , and {f}_{b i o m a s s} = \frac{\partial f}{\partial {c}_{b i o m a s s}}$$

To simplify notation,

$$let α = \frac{1}{ϕ} ( {f}_{b i o m a s s} {c}_{b i o m a s s}^{i n} + {\sum }_{j} {f}_{j} {c}_{j}^{i n} ) and$$

$$β = \frac{1}{ϕ} ( {f}_{b i o m a s s} μ + {\sum }_{j} {f}_{j} {R}_{j} ) .$$

With this new notation,

$$\frac{dV}{df} = α {Q}^{i n} - {Q}^{out} + β V .$$

α describes how input media and bioreactor media affect the equation of state. If there is no difference in these effects, then α=1. In the last term, β describes how the chemistry in the bioreactor affects the equation of state. If chemistry has no effect on the equation of state, then β=0.

If we assume c_{biomass} is constant, then,

$$\frac{dV}{df} = μ V - {Q}^{out} .$$

Consequently, the volumetric inflow rate required to increase the bioreactor volume while keeping biomass concentration constant with time is given as

$${Q}^{i n} = \frac{μ - β}{α} V .$$

### Utilizing the Specific Volume Equation of State

The partial molar volumes are functions of temperature, pressure, and composition. If ideal mixing is assumed, then the partial molar volumes become specific volumes which are functions of only temperature and pressure. With the ideal mixing assumption, the equation of state becomes 1=c_{biomass}V_{biomass}^{∘}+ΣⱼcⱼVⱼ^{∘}, where V_{biomass}^{∘} is the specific volume of pure biomass and Vⱼ^{∘} is the specific volume of pure component j.

We assume that the equation of state is f=c_{biomass}V_{biomass}^{∘}+ΣⱼcⱼVⱼ^{∘}−1, with fⱼ=Vⱼ^{∘}, and f_{biomass}=V_{biomass}^{∘}. Consequently, α=ΣⱼVⱼ^{∘}cⱼⁱⁿ and β=V_{biomass}^{∘}μ+ΣⱼVⱼ^{∘}Rⱼ. The volumetric inflow rate to maintain constant biomass is given as

$${Q}^{i n} = \frac{μ}{φ} ( V - {V}_{b i o m a s s} ) - \frac{V}{φ} {\sum }_{j} {V}_{j}^{∘} {R}_{j} where φ = {\sum }_{j} {V}_{j}^{∘} {c}_{j}^{i n} .$$

### Utilizing the Partial Molar Volume Equation of State

$${Q}^{i n} = \frac{μ}{φ} ( V - {V}_{b i o m a s s} ) - \frac{V}{φ} {\sum }_{j} {V}_{j}^{∘} {R}_{j}$$

can be generalized further by using partial molar volumes instead of specific volumes for the equation of state. Using partial molar volumes for the equation of state gives

$$f = {c}_{b i o m a s s} {\overline{V}}_{b i o m a s s} + {\sum }_{j} {c}_{j} {\overline{V}}_{j} - 1 with$$

$${f}_{j} = {\overline{V}}_{j} \frac{\partial {\overline{V}}_{j}}{\partial {c}_{j}} + {c}_{b i o m a s s} \frac{\partial {\overline{V}}_{b i o m a s s}}{\partial {c}_{b i o m a s s}} + {\sum }_{i \neq j} {c}_{i} \frac{\partial {\overline{V}}_{i}}{\partial {c}_{j}} and$$

$${f}_{b i o m a s s} = {\overline{V}}_{b i o m a s s} \frac{\partial {\overline{V}}_{b i o m a s s}}{\partial {c}_{b i o m a s s}} + {\sum }_{j} {c}_{j} \frac{\partial {\overline{V}}_{j}}{\partial {c}_{b i o m a s s}} .$$

From this, α, β, and Qⁱⁿ can be calculated and simplified if it is known that the partial molar volumes for a given media component is independent of a set of media components.

From this more detailed analysis, we conclude once again that the the concept of a variable-volume chemostat is viable. Modern computer-controlled pumps can definitely adjust their pumping rate with sufficiently accuracy and speed to be able to track and account for the increase in media volume within the chemostat as a future sample is accumulated and stored within the chemostat. This approach eliminates the sources of error and uncertainty that arise from attempting to collect samples whose volumes represent significant fraction of the chemostat volume and whose biochemistry can be difficult to halt as cells are collected over long periods of time. This will become particularly important as instruments are built that contain a large number of very small chemostats.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

What is claimed is:

1. A chemostat, comprising:
a bioreactor chamber containing media with cells for cell growth and cell division;
an input tube coupled to the chamber for delivering nutrient-laden media that support the cell growth and the cell division within the chamber, at an inflow rate; and
an output tube coupled to the chamber for withdrawing a sample from the chamber at an outflow rate,
wherein the inflow rate and the outflow rate are regulated such that the chemostat is operable in a sample accumulation phase or a sample withdrawal phase immediately following the sample accumulation phase, wherein in the sample accumulation phase, the outflow rate is zero and the inflow rate increases in proportion to an instantaneous volume of media in the chamber so as to accumulate the sample without changes in a metabolic state within the chamber; and wherein in the sample withdrawal phase, the outflow rate is regulated at a higher rate than the inflow rate to withdraw the accumulated sample from the chamber rapidly at one time and the inflow rate remains in proportion to the instantaneous volume to maintain chemostasis in the chamber.

2. The chemostat of claim 1, being a variable-volume chemostat in which the instantaneous volume of media varies with time.

3. The chemostat of claim 2, wherein the quantity of nutrients per cell within the media in the chamber remains unchanged.

4. The chemostat of claim 1, being configured such that a dilution rate that is a ratio of the inflow rate divided by the instantaneous volume is a constant, so as to maintain the same ratio of nutrient delivery per cell independent of the total volume of the media and the cells that it contains.

5. The chemostat of claim 1, wherein a maximum volume of the sample that is accumulated and then removed is determined by a difference between a maximum allowable volume and a minimum allowable volume of the chamber, and the time required to accumulate the sample is determined by the maximum volume of the sample divided by the inflow rate.

6. The chemostat of claim 1, wherein the inflow rates and the outflow rate are adjustable simultaneously to support different phases of sample accumulation and sample withdrawal.

7. The chemostat of claim 1, wherein the inflow rate and the outflow rates are of different functions of time such that a difference between the inflow rate and the outflow rate equals a rate of change of the instantaneous volume with time.

8. The chemostat of claim 7, wherein when the inflow rate is greater than the outflow rate, then the volume of fluid within the chemostat increases with time, and when the outflow rate is greater than the inflow rate, the volume of fluid decreases in time.

9. The chemostat of claim 1, being operable in a filling phase during which the outflow rate is zero and the inflow rate is greater than zero so that the instantaneous volume increases with time, or in a steady-state phase during which the outflow rate is same as the inflow rate.

10. The chemostat of claim 1, further comprising an output pump coupled to the output tube for regulating the outflow rate, and an input pump coupled to the input tube for regulating the inflow rate.

11. The chemostat of claim 10, wherein the output pump is turned off at the beginning of the sample accumulation phase and turned on at the beginning of the sample withdrawal phase.

12. The chemostat of claim 1, further comprising a means for stirring and oxygenating the media in the chamber respectfully at a stirring rate and a gas exchange rate to ensure that the media within the chamber is well mixed, uniformly oxygenated, and at the desired pH over the full range of volumes of media contained in the bioreactor chamber during all phases of operation.

13. The chemostat of claim 12, wherein the input gas mixture composition, the gas exchange rate, and the stirring rate are adjustable so as to ensure that the local conditions throughout the bioreactor chamber remain unchanged over the full range of volumes of media contained in the bioreactor chamber during all phases of operation.

14. The chemostat of claim 12, wherein growth conditions, and nutrient and gas concentrations within the entire media in the chamber are maintained at original conditions by modulating the inflow rate, the outflow rate, the gas exchange rate and the stirring rate in a manner that maintains static biochemical conditions independent of the instantaneous volume of media and cells within the chamber.

15. The chemostat of claim 12, wherein a fraction of the media in the chamber is withdrawable without prior accumulation, but with the inflow rate, the outflow rate, the gas exchange rate and the stirring rate modulated post-withdrawal in a manner that maintains static biochemical conditions independent of the instantaneous volume of cells and media within the chamber.

16. The chemostat of claim 12, wherein by the dynamic control of the inflow rate, the outflow rate, the gas exchange rate, and the stirring rate, any arbitrary volume within the chamber can be maintained at the same biochemical state as any volume within an industry-standard, constant-volume chemostat is achievable.

17. A method for operating a chemostat, wherein the chemostat is characterized with a media volume that varies with time in a chamber, an inflow rate at which nutrient-laden media are delivered into the chemostat, and an outflow rate at which a sample is withdrawn from the chemostat, comprising:
regulating the inflow rate, the outflow rate, and the gas mixture such that the chemostat operates in a sample accumulation phase or a sample withdrawal phase immediately following the sample accumulation phase,
wherein in the sample accumulation phase, the outflow rate is zero or very small and the inflow rate increases in proportion to an instantaneous volume of media in the chamber so as to accumulate the sample without changes in a metabolic state within the chamber; and
wherein in the sample withdrawal phase, the outflow rate is regulated at a higher rate than the inflow rate to withdraw the accumulated sample from the chamber rapidly at one time and the inflow rate and gas exchange rate remains in proportion to the instantaneous volume to maintain chemostasis in the chamber.

18. The method of claim 17, wherein a dilution rate that is a ratio of the inflow rate divided by the instantaneous volume is a constant, so as to maintain the same ratio of nutrient delivery per cell independent of the total volume of the media and cells.

19. The method of claim 17, further comprising stirring and oxygenating the media in the chamber respectfully at a stirring rate and a gas exchange rate to ensure that the media within the chamber is well mixed, uniformly oxygenated, and has the desired carbon dioxide levels.

20. The method of claim 19, wherein the gas exchange rate and the stirring rate are adjustable so as to ensure that the local conditions throughout the bioreactor chamber remain unchanged.
